(19) United States
(12) Patent Application Publication
(10) Pub. No.: US 2009/0220626 A1
(43) Pub. Date: Sep. 3, 2009

(54) GRAPE SEED EXTRACT AND ITS CONSTITUENTS FOR USE AS CYP2A6 INHIBITORS

(75) Inventors: James Madison Mathews, Durham, NC (US); Amy Sue Etheridge, Cary, NC (US)

Correspondence Address: ROTHWELL, FIGG, ERNST & MANBECK, P.C., 1425 K STREET, N.W., SUITE 800, WASHINGTON, DC, 20005, US

(73) Assignees: Nicogen Imc., Toronto. Ontario, CA (US); Research Triangle Institute, Research Triangle Park, NC (US)

(21) Appl. No.: 12/089,466

(22) PCT Filed: Oct. 6, 2006
(86) PCT No.: PCT/CA06/01642

§ 371 Date: Oct. 28, 2008

Related U.S. Application Data
(60) Provisional application No. 60/723,919, filed on Oct. 6, 2005.

(51) Int. Cl. A61K 36/87; A61P 35/00; A61P 9/00; A61P 25/34; A61P 11/00

(52) U.S. Cl. 424/766

(57) ABSTRACT

The present invention relates to a use of grape seed extract or its constituents to treat conditions which benefit from an inhibition of cytochrome P450 2A6 (CYP2A6). In particular conditions which benefit from the inhibition of CYP2A6 are conditions which benefit from decreased metabolism of nicotine to cotinine, for example, preventing, reducing or treating smoking, and conditions which benefit from a decrease in the in vivo formation of carcinogens, for example prevention or treatment of cancer.

### FIELD OF THE INVENTION

The present invention relates to inhibitors of the enzyme CYP2A6, to pharmaceutical and diagnostic compositions containing them and to their medical use, particularly in the treatment, reduction and/or prevention of disorders associated with nicotine and tobacco use, dependence, and other indications for nicotine, such as diseases being treated by nicotine (i.e. Parkinson's disease). In particular the present invention relates to the use of grape seed extract for the treatment of conditions which benefit from an inhibition of CYP2A6.

### BACKGROUND OF THE INVENTION

Tobacco is an insidious chemical product as, among its numerous toxic compounds, it contains nicotine which is responsible for the dependency associated with tobacco smoking (Khuranas, S. et al., Respir. Med. 2003, 97, 295-301). According to the World Health Organization (WHO), tobacco use is the leading cause of the disease burden measured in disability adjusted life years in developed countries and one of the top 10 health risk factors even in the poorest developing regions (Ezzati, M. et al., Lancet 2003, 362, 271-280).

Nicotine is the essential component smokers seek from tobacco use. Various nicotine preparations have been developed as medication to assist in smoking cessation, and nicotine has also been evaluated in the treatment of a variety of medical disorders including Alzheimer's, Parkinson's Tourettes syndrome, sleep apnea, and attention deficit disorder. (Benowitz, N. L., Annu. Rev. Pharmacol. Toxicol. 1996, 36, 597-613). Nicotine is eliminated primarily by metabolism of nicotine to inactive cotinine. The main enzyme catalyzing this reaction is cytochrome P450 2A6 (CYP2A6) (Messina, E. S. et al., J Pharmacol Exp Ther 1997, 282, 1608-1614; Oscarson, M., Drug Metab. Dispos. 2001, 29, 91-95; Raunio, H. et al., Br. J. Clin. Pharmacol. 2001, 52, 357-363; Sellers, E. M. et al., Drug Discovery Today 2003, 8, 487-493). Individuals having deficient CYP2A6 enzyme function, due to inactive alleles of the CYP2A6 gene, display a decreased capacity for nicotine metabolism and these individuals may be less likely to become smokers, are less prone to nicotine addiction, smoke less per day and for shorter durations than individuals with a normally functioning CYP2A6 enzyme (Kitagawa, K., Biochem. Biophys. Res. Commun. 1999, 262, 146-151; Inoue, K. et al., Arch. Toxicol. 2000, 73, 532-539; Nakajima, M. et al. Clin. Pharmacol. Ther. 2000, 67, 57-69; Tyndale, R. F. et al. Ther. Drug Monit. 2002, 24, 163-171). Pilot studies have found that chemical inhibition of the CYP2A6 enzyme can reduce the smoking frequency (Sellers, E. M. et al., Clin. Pharmacol. Ther. 2000, 68, 35-43; Tyndale, R. F. et al. Ther. Drug Monit. 2002, 24, 163-171; Sellers et al. Nicotine Tob Res 2003, 5: 891-9). There has also been reported evidence that in the Japanese population, those individuals with inactive CYP2A6 alleles are protected from developing lung cancer caused by cigarette smoking (Ando M. et al. J. Epidemiol. 2003; 13: 176-81; Ariyoshi N. et al. Cancer Epidemiol Biomarkers Prev. 2002; 11(9):890-4; Miyamoto M. et al. Biochem. Biophys. Res. Commun. 1999, 261, 658-660) and smoke less (Fujieda et al. Carcinogenesis 2004; 25(12):2451-8).

Other than nicotine dependence as a result of tobacco use, nicotine itself is not considered hazardous, namely it is not considered to be a causative agent in cancer and heart and lung disease. It is the other products which are found in tobacco products which are considered to be harmful, including combustion products such as carbon monoxide, gases and tar.

Nicotine is routinely used in smoking cessation therapy in which nicotine is delivered to individuals in an attempt to assist that individual in abstaining from tobacco products. In current smoking cessation therapy, nicotine is administered to an individual as chewing gum, transdermal patches, or via nasal spray. To the knowledge of the inventors, oral nicotine administration is not currently commercially available for the reason that oral nicotine must first pass through the liver before entering the systemic circulation. As a result, very high degree of CYP2A6 mediated first pass metabolism occurs in the liver resulting in a small fraction of the nicotine reaching the blood and brain. Since oxidation by CYP2A6 is the rate-limiting step in nicotine inactivation, blocking this reaction by a chemical inhibitor would increase nicotine bioavailability and allow for peroral administration of nicotine (Sellers, E. M. et al. Drug Discovery Today 2003, 8, 487-493). CYP2A6 inhibitors can also be used to reduce nicotine inactivation from alternative sources of nicotine, such as smoking, resulting in reduced smoking or to reduce the inactivation of nicotine from non-oral nicotine replacement therapies thus increasing nicotine duration and bioavailability. In addition to nicotine inactivation, the human CYP2A6 and mouse CYP2A5 enzymes can also activate several other xenobiotics, such as many of the tobacco-specific nitrosamines and other toxic compounds (Oscarson, M. Drug Metab. Dispos. 2001, 29, 91-95; Raunio, H. et al., Br. J. Clin. Pharmacol. 2001, 52, 357-363; Tyndale, R. F. et al., Drug Metab. Dispos. 2001, 29, 548-552). Pilot data has demonstrated that CYP2A6 inhibitors in vivo can reduce the activation of tobacco-smoke nitrosamines, rerouting these nitrosamines to detoxified glucuronidated products (Sellers et al. Nicotine Tob Res 2003, 5:1-9).

Since nicotine is inactivated by metabolism to cotinine by the CYP2A6 enzyme, understanding of the inhibitory structure-activity of the CYP2A6 enzyme is crucial. To the knowledge of the inventors, about 200 compounds have been tested to date for the inhibitory properties on CYP2A5 or CYP2A6 enzymes. However, there is still a need for identifying potent and specific chemical inhibitors of the CYP2A enzymes, in particular the human CYP2A6 enzyme, as these inhibitors can be used to regulate nicotine metabolism in an individual.

Grape seed extract is a popular herbal supplement sold by food, drug and mass-market retail outlets. Common uses for grape seed extract include the treatment and prevention of cardiovascular conditions, including atherosclerosis, peripheral vascular disease and myocardial or cerebral infarction. The chemical class proanthocyanidins, including catechin, epicatechin, epigallocatechin and epigallocatechin gallate, are among the most abundant phenolic compounds in grape seeds, and are thought to provide the principal pharmacologically active constituents (Santos-Buelga C. et al., J. Food Sci. Agric. 2000, 80, 1094-1117). Standardized grape seed extracts are reported to contain 76% oligomeric procyanindin as reported by Vinson et al. (Mol. Cell Biochem. 2002, 240(1-2), 99-103) or 89% proanthocynidin as reported by Sugisawa et al. (Pharm. Bull. 2004, 27(9), 1459-61) depending on the source of the grape seed extract. In vitro, proanthocyanidins have been shown to have strong antioxidant activity and to scavenge reactive oxygen and nitrogen species (Arteel G. E., et al., FEBS Lett 1999, 462, 167-170; Ariga T., et al., Agric. Biol. Chem. 1990 54, 2499-2504; Hagerman A. E., et al., J. Agric. Food Chem. 1998, 46, 1887-1892). In vivo studies in rat have shown that proanthocyanidins inhibit the progression of atherosclerosis (Yamakoshi J., et al., Atherosclerosis 1999, 142, 139-149) and prevent the increase of LDL cholesterol associated with a high-cholesterol diet (Tebib K., et al., Enzyme Protein 1994, 48, 51-60). Additionally, epidemiologic data has demonstrated an inverse correlation between dietary intake of proanthocyanidins and coronary heart disease, stroke and cancer (Hertog M. G. L., et. al., Lancet 1993, 342, 1007-1011, Hertog M. G. L., et. al., Arch. Intern. Med. 1995, 155, 381-386; Keli S. O., et al., Arch. Intern. Med. 1996, 156, 637-642; Knekt P. et al., Br. Med. J. 1996, 312, 478-481).

Despite the potential health benefits of proanthocyanidins and the popularity of grape seed extract, data on its metabolism, disposition and toxicokinetics and in particular, its effects on human hepatic P450 2A6 enzyme and cellular transport has previously been lacking.

### SUMMARY OF THE INVENTION

It has been found that grape seed extract and its constituents are inhibitors of the enzyme CYP2A6.

According to a first aspect of the present invention there is included a method of preventing or treating conditions which benefit from the inhibition of CYP2A6 by administering grape seed extract or one or more of its constituents to a subject in need thereof.

The present invention also relates to a use of grape seed extract or one or more of its constituents to prevent or treat conditions which benefit from an inhibition of CYP2A6. Further, the present invention relates to a use of grape seed extract or one or more of its constituents to prepare a medicament to prevent or treat conditions which benefit from an inhibition of CYP2A6.

In embodiments of the invention the condition which benefits from an inhibition of CYP2A6 is one which benefits from an inhibition of the metabolism of nicotine to cotinine. In embodiments of the invention the conditions which benefit from an inhibition of metabolism of nicotine to cotinine include nicotine-use disorders, nicotine-induced disorders, and/or nicotine-related disorders. Examples of nicotine-use disorders include 1) dependent and non-dependent tobacco use (i.e. smoking) and related pulmonary and cardiovascular diseases and 2) other drug dependencies. Examples of nicotine-induced disorders include withdrawal from dependent and non-dependent tobacco use. Examples of nicotine related disorders include opioid related disorders, proliferative diseases, cognitive disorders, neurological disorders and mental disorders and more specifically ulcerative colitis, Alzheimer's disease, Parkinson's disease, Tourette's syndrome, sleep apnea, attention deficit disorders, psychoses, schizophrenia, anxiety, depression, alcoholism, opiate dependence, memory deficits, cholinergic deficits and the like. In further embodiments of the invention the condition which benefits from inhibition of CYP2A6 is one that is the result of activation of various chemical species by CYP2A6 for example a condition that results from the activation of procarcinogens such as tobacco related nitrosamines and other toxic compounds. An example of a condition which benefits from the inhibition of the activation of procarcinogens is cancer, and more specifically colorectal and lung cancer.

The present invention also relates to a method of enhancing nicotine replacement therapy comprising administering an effective amount of grape seed extract or one or more of its constituents to a subject in need thereof. The invention also relates to a use of grape seed extract or one or more of its constituents to enhance nicotine replacement therapy and to a use of grape seed extract or one or more of its constituents to prepare a medicament to enhance nicotine replacement therapy. The nicotine replacement therapy being enhanced may be oral therapy, or therapy via transdermal patch, nasal, spray or chewing gum or other nicotine delivery systems such as inhalers. In an embodiment of the invention, the grape seed extract or one or more of its constituents is administered contemporaneously with nicotine.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 shows the specification sheet for a commercially produced grape seed extract.

FIG. 2 shows the certificate of analysis of a commercially produced grape seed extract.

FIG. 3 shows HPLC chromatograms of catechin standard, grape seed extract and grape seed extract fortified with catechin.

FIG. 4 is a graph showing the activity of various CYP450 enzymes following incubation of human hepatic microsomes with grape seed extract.

FIG. 5 is a graph showing the activity GSE and Capsule extracted in either buffer or methanol in mouse liver microsomes.

FIG. 6 is a graph showing the activity of GSE and Capsule extracted in either buffer or methanol in CYP2A6 Supersomes.

FIG. 7 is graph showing the activity of cytisine in CYP2A6 Supersomes

### DETAILED DESCRIPTION OF THE INVENTION

Substances showing inhibition of the enzyme CYP2A6 have been identified. In particular the substances identified are grape seed extract and its constituents. These substances are useful for preventing or treating diseases which benefit from a decrease in the metabolism of nicotine or a decrease the formation of carcinogens.

The term “grape seed extract” as used herein refers to the material obtained via extraction of grape seeds. In one embodiment of the invention grape seed extract is obtained by extraction of dry grape seeds with hot water to obtain the polyphenolic compounds. In a further embodiment the polyphenolic compounds may be purified following extraction. In still a further embodiment, purification of the polyphenolic compounds may be achieved by filtration. In yet another embodiment of the invention, polyphenolic extracts are purified using food grade membranes and adsorption resins as described in U.S. Pat. No. 5,912,363 (which is hereby incorporated by reference). In a suitable embodiment of the invention the grape seed extract contains over 80 percent polyphenolic compounds. In a specific embodiment of the invention, grape seed extract may be obtained from a commercial source, for example Activin® from San Joaquin Valley Concentrates of Fresno, Calif.

The terms “constituents of grape seed extract” and “constituents” as used herein refer to any one or more compounds, or pharmaceutically acceptable solvates and prodrugs thereof, found in grape seed extract having CYP2A6 inhibitory activity. These compounds may be isolated directly from grape seed extract or may be isolated from other sources or prepared synthetically. Grape seed extract is generally known to contain antioxidant compounds. More particularly grape seed extract is known to contain, as active ingredients, polyphenolic compounds belonging to the flavanol family, which is a subclass of flavanoids. These polyphenolic compounds are generally known as catechins and include the compounds catechin, epigallocatechin, (EGC), epigallocatechin gallate (EGCG), and epicatechin (EC), which are shown in Scheme 1. In an embodiment of the invention the constituent is a catechin.

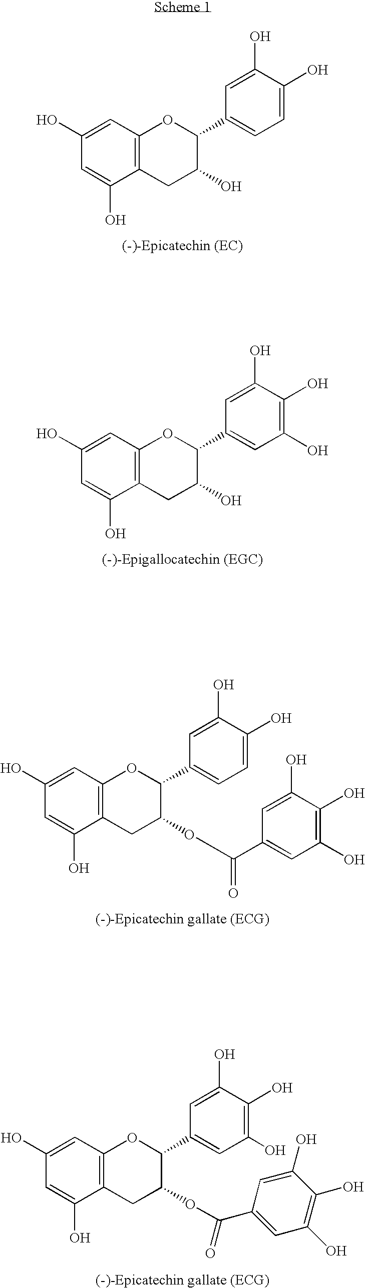

It is to be clear that the present invention also includes method, uses and compositions comprising pharmaceutically acceptable solvates and/or prodrugs of the one or more constituents of grape seed extract.

The one or more constituents of grape seed extract may have at least one asymmetric centre. Where the constituent possesses more than one asymmetric centre, it may exist as diastereomers. It is to be understood that all such isomers and mixtures thereof in any proportion are encompassed within the scope of the present invention. It is to be understood that while the stereochemistry of the one or more constituents may be as provided for in any given compound listed herein, such compounds may also contain certain amounts (e.g. less than 20%, preferably less than 10%, more preferably less than 5%) of compounds having alternate stereochemistry.

The term “pharmaceutically acceptable” means compatible with the treatment of animals, in particular, humans.

The term “solvate” as used herein means the incorporation of molecules of a suitable solvent into the crystal lattice of a compound. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

The term “prodrug” as used herein will be functional derivatives of the compound which are readily convertible in vivo into the compound from which it is notionally derived. Prodrugs may be conventional esters formed with, for example, available hydroxy, or amino groups. For example, an available OH may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs are those in which one or more of the hydroxy groups in the compounds is masked as groups which can be converted to hydroxy groups in vivo. Conventional procedures for the selection and preparation of suitable prodrugs are described, for example, in “Design of Prodrugs” ed. H. Bundgaard, Elsevier, 1985.

The present invention includes the use of grape seed extract and its constituents in therapeutic methods and compositions for inhibiting CYP2A6. In particular, the present invention includes the use of grape seed extract and its constituents as a medicament.

Accordingly the present invention includes a method of preventing or treating conditions which benefit from an inhibition of CYP2A6 comprising administering an effective amount of grape seed extract or one or more of its constituents to a subject in need thereof.

The present invention also relates to a use of grape seed extract or one or more of its constituents to prevent or treat conditions which benefit from an inhibition of CYP2A6. Further, the present invention relates to a use of grape seed extract or one or more of its constituents to prepare a medicament to prevent or treat conditions which benefit from an inhibition of CYP2A6.

To “inhibit” or “suppress” or “reduce” a function or activity, such as CYP2A6 activity, is to reduce the function or activity when compared to otherwise same conditions except for a condition or parameter of interest, or alternatively, as compared to another condition. The term “inhibition”, in the context of the present invention, are intended to have a broad meaning and encompass agents which directly or indirectly (e.g., via reactive intermediates, metabolites and the like) act on CYP2A6 to inhibit or otherwise regulate the ability of CYP2A6 to catalyze metabolism of nicotine to cotinine.

The term “inhibitor” or synonyms thereof, as used herein refers to grape seed extract and/or one or more of its constituents.

The term a “therapeutically effective amount”, “effective amount” or a “sufficient amount” of grape seed extract, or one or more of its constituents, as used herein is a quantity sufficient to, when administered to the subject, including a mammal, for example a human, effect beneficial or desired results, including clinical results, and, as such, an “effective amount” or synonym thereto depends upon the context in which it is being applied. For example, in the context of inhibiting CYP2A6, for example, it is an amount of grape seed extract, or one or more of its constituents, sufficient to achieve such an inhibition in CYP2A6 activity as compared to the response obtained without administration of the substance. In the context of disease, therapeutically effective amounts of grape seed extract, or one or more of its constituents, are used to treat, modulate, attenuate, reverse, or effect conditions which benefit from an inhibition of CYP2A6 in a subject. An “effective amount” is intended to mean that amount of grape seed extract, or one or more of its constituents, that is sufficient to treat, prevent or inhibit conditions which benefit from an inhibition of CYP2A6 or a disease associated with conditions which benefit from an inhibition of CYP2A6. The amount of grape seed extract, or one or more of its constituents, that will correspond to such an amount will vary depending upon various factors, such as the given extract or constituent, the pharmaceutical formulation, the route of administration, the type of disease or disorder, the identity of the subject, and the like, but can nevertheless be routinely determined by one skilled in the art. Also, as used herein, a “therapeutically effective amount” of grape seed extract, or one or more of its constituents, is an amount which prevents, inhibits, suppresses or reduces CYP2A6 activity (e.g., as determined by clinical symptoms) in a subject as compared to a control. As defined herein, a therapeutically effective amount of a grape seed extract, or one or more of its constituents, may be readily determined by one of ordinary skill by routine methods known in the art.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, regulation of a disease and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or the time course of the progression is slowed or lengthened, as compared to not treating the disorder.

The term “prevention” or “prophylaxis”, or synonym thereto, as used herein refers to a reduction in the risk or probability of a subject becoming afflicted with a condition which benefits for the inhibition of CYP2A6 or manifesting a symptom associated with a condition which benefits from the inhibition of CYP2A6.

The term “subject” as used herein includes all members of the animal kingdom including human. The subject is preferably a human.

In embodiments of the invention a condition which benefits from an inhibition of CYP2A6 is one which benefits from an inhibition of the metabolism of nicotine to cotinine, including nicotine use disorders, nicotine-induced disorders and/or nicotine related disorders.

The present invention further relates to a method of preventing or treating a condition requiring inhibition of nicotine metabolism to cotinine comprising administering an effective amount of grape seed extract or one or more of its constituents to a subject in need thereof. The invention also relates to a use of grape seed extract or one or more of its constituents to prevent or treat a condition requiring inhibition of nicotine metabolism to cotinine and a use of grape seed extract or one or more of its constituents to prepare a medicament to prevent or treat a condition requiring inhibition of nicotine metabolism to cotinine.

In one aspect, the nicotine-use disorder is dependent or non-dependent tobacco use or smoking. Accordingly, the present invention provides a method of preventing, treating or regulating smoking comprising administering an effective amount of grape seed extract or one or more of its constituent to a subject in need thereof. Also included is a use of grape seed extract or one or more of its constituent to prevent, treat or regulate smoking and a use grape seed extract or one or more of its constituents to prepare a medicament to prevent, treat or regulate smoking.

As used throughout this specification, the terms “smoking prevention” and “preventing smoking”, are intended to mean that the likelihood of the onset of smoking (i.e., the progression from a cigarette to regular smoking) in a current non-smoking individual (i.e., a person who has never smoked or is a ex-smoker) and the return to smoking of a previous smoker (i.e. relapse prevention) is substantially reduced.

The terms “smoking regulation” and “regulating smoking”, as used throughout this specification, are intended to mean that the amount smoked by a current smoking individual is reduced or, at least, fails to increase.

The terms “smoking treatment” or “treatment of smoking” means the stopping of all smoking (i.e. smoking cessation) or smoking reduction as reflected in less use of tobacco products, a decrease in pattern of use and/or a decrease in tobacco smoke exposure, or reduction of the amount of smoking leading to cessation. The measure of tobacco smoke exposure can be measured by analyzing breath carbon monoxide.

By preventing treating and/or regulating smoking in an individual, the method of the present invention also treats related pulmonary and cardiovascular diseases associated with smoking and other drug dependence.

An oral nicotine replacement therapy containing nicotine alone would be ineffective due to the extensive metabolism of nicotine in the liver which significantly decreases the systemic availability of the nicotine. However, administering the nicotine with a CYP2A6 inhibitor would increase the bioavailability and the effectiveness of the oral nicotine therapy.

Accordingly, the present invention also relates to a method of enhancing oral nicotine replacement therapy comprising administering an effective amount of grape seed extract or one or more of its constituents to a subject in need thereof. The invention also relates to a use of grape seed extract or one or more of its constituents to enhance oral nicotine replacement therapy and a use of grape seed extract or one or more of its constituents to prepare a medicament to enhance oral nicotine replacement therapy. In an embodiment of the invention, the grape seed extract or one or more of its constituents is administered contemporaneously with nicotine.

The term “enhancing oral nicotine replacement therapy” as used herein means to decrease the dose of any form of oral nicotine replacement therapy that is needed and/or to prolong the duration of action of the therapy and/or increase their effectiveness in the treatment of tobacco dependence.

As used herein, “administered contemporaneously” means that the two agents are administered to a subject such that they are both biologically active in the subject at the same time. The exact details of the administration will depend on the pharmacokinetics of the two substances in the presence of each other, and can include administering one substance within 24 hours, intermittently or as infrequent as weekly, of administration of the other, if the timecourse of enzyme inhibition is suitable. Design of suitable dosing regimens are routine for one skilled in the art. In particular embodiments, two agents will be administered substantially simultaneously, i.e. within minutes of each other, or in a single composition that comprises both substances.

Smoking conditions may develop with the use of all forms of tobacco (e.g. cigarettes, chewing tobacco, snuff, pipes and cigars) and with prescription medications (e.g. nicotine gum, nicotine patch, spray, pulmonary inhalation or other forms). In particular the treatment methods of the invention may be used to diminish a subject's desire to smoke and thereby alter smoking behaviour leading to smoking cessation or reduction smoking leading to smoking cessation. The inhibitor and nicotine combination can be used for the treatment of other neurological syndromes. The inhibitor alone or combined with nicotine can also be used for reducing smoking behaviours to reduce cardiovascular and/or pulmonary disease risk.

The treatment methods of the present invention by regulating nicotine metabolism in an individual are highly effective. The methods and compositions modify the behavioural components of smoking by reducing nicotine metabolism to cotinine. An individual with reduced nicotine metabolism following administration of grape seed extract or one or more of its constituents will alter smoking behaviour and smoke exposure because of modification of nicotine requirements. The methods and compositions of the invention are expected to show patterns of reduction, more sustained abstinence, and lower tobacco smoke exposure than obtained with prior art methods in particular those using nicotine deprivation.

The behavioural component of smoking is particularly important in some groups of individuals, and thus the methods, of the invention, in modifying behavioural components, may be particularly useful in reducing smoking in those individuals. For example, it has been found that behavioural components are significant in tobacco use by women. The present invention permits the development of behavioural learning on an individual/or group basis.

The treatment methods of the invention are also particularly suited to regulate nicotine metabolism in individuals or populations having high levels of CYP2A6. For example, Caucasians in North America have high levels of CYP2A6, (see U.S. Pat. No. 6,908,631).

The methods of the invention also have the advantage of individualization and flexibility in treatment duration. The compositions and treatment methods are particularly suitable for severely dependent individuals, previous treatment failures, individuals unable to accept the current approach of complete cessation, treatment/prevention of relapse, or concurrent treatment with other methods such as the nicotine patch. It is expected that the compositions and treatments of the invention will decrease the doses of nicotine patch and all other forms of nicotine replacement therapies that are needed and will prolong the duration of action of the therapy and/or increase their effectiveness in the treatment of tobacco dependence.

The methods of the invention used in treating individuals with nicotine use disorders and nicotine-induced disorders are also useful in the treatment and prophylaxis of other diseases or conditions, including nicotine-related disorders such as opioid related disorders; proliferative diseases; cognitive, neurological or mental disorders; pulmonary or cardiovascular diseases; and other drug dependencies in the individuals (See Benowitz N. L. Ann. Rev. Pharmacol. Toxicol. 1996, 36; 597-613). Examples of such underlying diseases or conditions include malignant disease, psychosis, schizophrenia, Parkinson's disease, Alzheimer's disease, Tourette's syndrome, anxiety, depression, alcoholism, opiate dependence, memory deficits, ulcerative colitis, cholinergic deficits, sleep apnea, attention deficit disorder, and the like.

The methods of the invention may also be used in the prophylaxis and treatment of individuals having other conditions which require an inhibition of CYP2A6. For example, CYP2A6 is known to activate several procarcinogens such as NNK (Crespi C L, et al., “A tobacco smoke-derived nitrosamine, 4-(methylnitrosamino)-1-(3-pyridyl)-1-butanone, is activated by multiple human cytochrome P450s including the polymorphic human cytochrome P4502D6,” Carcinogenesis, 12(7):1197-201 (1991)); aflaxtoxin B1 (Yun C H, et al., “Purification and characterization of human liver microsomal cytochrome P4502A6,” Molec. Pharmacol., 40(5):679-85 (1991)); hexamethylphosphoramide (Ding X, et al., “Mossbauer studies on the metal-thiolate cluster formation in Fe(II)-metallothionein,” Eur. J Biochem., 171(3):711-4 (1988)), and nitrosodimethylamine (Davies R L, et al., “Development of a human cell line by selection and drug-metabolizing gene transfection with increased capacity to activate promutagens,” Carcinogenesis, 10:885-891 (1989); Fernandez-Salguero, et al. Am J Hum Genet, 57:651-60 (1995); Fernandez-Salguero, et al. Pharmacogenetics, 5:S123-8 (1995)). Therefore, inhibitors of CYP2A6 may be useful in the prophylaxis (e.g., inhibition of CYP2A6 substrates thereby decreasing genotoxicity, cytotoxicity and/or mutagenicity) and treatment of malignant diseases, and, without limitation, the above-mentioned conditions and diseases. Altered CYP2A6 genotypes have also been associated with a decrease risk for developing colorectal cancer (Sweeney, et al Toxicology 181-182: 83-87 (2002); Sachse, et al. Carcinogenesis, 23:1839-1849 (2002); Nowell, et al. Mutation Research, 506-507:175-178 (2002); Nowell et al. Cancer Epidemiology, Biomarkers and Prevention, 11:377-383 (2002)) and lung cancer (Fujieda et al. Carcinogenesis 2004; 25(12):2451-8). Accordingly, inhibition of CYP2A6 can also be used for the treatment and/or prevention of colorectal cancer and lung cancer.

Therefore, in another aspect, the present invention includes a method of regulating the formation of a carcinogen in subject in need thereof comprising administering an effective amount of grape seed extract or one or more of its constituents. Also included is a use of a grape seed extract or one or more of its constituents to regulate the formulation of a carcinogen and a use of grape seed extract or one or more of its constituent to prepare a medicament to regulate the formulation of a carcinogen.

The terms “carcinogen formation regulation” and “regulating formation of a carcinogen”, as used throughout this specification, are intended to mean that the occurrence of carcinogen formation in an individual is reduced. This may be achieved, for example, by using CYP2A6 inhibition to inhibit activation of procarcinogens present in the individual. As used throughout this specification, the term “procarcinogen” is meant to encompass any substance which is at least one of procytotoxic, promutagenic and progenotoxic (“pro” means the metabolite is more active than the parent compound).

In a further aspect, the present invention provides a method of preventing or treating cancer in a subject in need thereof comprising administering an effective amount of grape seed extract or one or more or its constituents. Also included is a use of grape seed extract or one or more of its constituents to prevent or treat cancer and a use of grape seed extract or one or more of its constituents thereof to prepare a medicament to prevent or treat cancer.

The terms “cancer prevention or treatment” and “preventing or treating cancer”, as used throughout this specification, are intended to mean that the likelihood of the onset of cancer in a current cancer-free individual (i.e., a person who has never had cancer or whose cancer is in remission) is substantially mitigated. They also mean that the reduction of carcinogen activation can prevent the reoccurrence of the cancer in subjects. In embodiment of the invention, the cancer is colorectal cancer.

The grape seed extract or its constituents may be tested for their ability to inhibit CYP2A6 using known methods, for example as described in PCT Patent Application WO 99/27919 or in Hickman D. et al. Drug Metab. Dispos. 1998, 26; 207-215.

Grape seed extract or its constituents are suitably formulated into pharmaceutical compositions for administration to human subjects in a biologically compatible form suitable for administration in vivo. Accordingly, the present invention further includes a pharmaceutical composition for treating conditions which benefit from an inhibition of CYP2A6 comprising an effective amount of grape seed extract or one or more of its constituents and a pharmaceutically acceptable carrier and/or diluent.

The compositions containing grape seed extract or one or more of its constituents can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (2000-20th edition) and in The United States Pharmacopeia: The National Formulary (USP 24 NF19) published in 1999). On this basis, the compositions include, albeit not exclusively, solutions of the active ingredients in association with one or more pharmaceutically acceptable vehicles or diluents, and contained in buffered solutions with a suitable pH and iso-osmotic with the physiological fluids.

The grape seed extract or its constituents may be used in any form including, in the form of solvates and/or prodrugs. All forms are within the scope of the invention.

In accordance with the methods of the invention, the compositions may be administered to a subject in a variety of forms depending on the selected route of administration, as will be understood by those skilled in the art. The compositions of the invention may be administered, for example, by oral, parenteral, buccal, sublingual, nasal, rectal, patch, pump or transdermal (topical) administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

The grape seed extract or constituent thereof may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the grape seed extract or one or more of its constituents may be incorporated with excipient and used in the form of ingestible tablets, buccal tablets, troches, capsules, elixirs, suspensions, syrups, wafers, and the like.

The grape seed extract or constituent thereof may also be administered parenterally. Solutions can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2000-20th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists. Ampoules are convenient unit dosages.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Compositions for topical administration may include, for example, propylene glycol, isopropyl alcohol, mineral oil and glycerin. Preparations suitable for topical administration include liquid or semi-liquid preparations such as liniments, lotions, applicants, oil-in-water or water-in-oil emulsions such as creams, ointments or pastes; or solutions or suspensions such as drops. In addition to the aforementioned ingredients, the topical preparations may include one or more additional ingredients such as diluents, buffers, flavouring agents, binders, surface active agents, thickeners, lubricants, preservatives, e.g. methyl hydroxybenzoate (including anti-oxidants), emulsifying agents and the like.

Sustained or direct release compositions can be formulated, e.g. liposomes or those wherein the active compound is protected with differentially degradable coatings, such as by microencapsulation, multiple coatings, etc. It is also possible to freeze-dry the grape seed extract or one or more of its constituents and use the lypolizates obtained, for example, for the preparation of products for injection.

The dosage administered will vary depending on the use and known factors such as the pharmacodynamic characteristics of the particular substance, and its mode and route of administration; age, health, and weight of the individual recipient; nature and extent of symptoms, kind of concurrent treatment, frequency of treatment, and the effect desired.

In some instances, instead of increasing the dosage of a compound, the kinetics of inhibition created by certain chemical compounds can be altered or enhanced by adding to the treatment protocol a second inhibitor to a substance (e.g., enzyme) that is capable of inhibiting the metabolism of the CYP2A6 inhibitor. By adding such a second inhibitor, the quantity of the CYP2A6 inhibitor will be maintained thus prolonging the beneficial effect of maintaining an elevated plasma concentration of nicotine. The use of such a second inhibitor is very beneficial since it facilitates treatment of individuals by maintaining substantially constant nicotine levels and acting locally on the kinetics of the CYP2A6 inhibitor. By using this approach, large dosages of centrally active compounds can be avoided.

Similarly, preexposure of an individual to an inhibitory substance sometimes can result in an inhibitory effect that will outlast the presence of the drug in the plasma or that will have a persistent effect in the individual despite the inhibitor's half life in the plasma. This phenomenon caused by preincubation or preexposure of an inhibitory substance can help increase the dose interval at which a dosage of the substance must be administered, decrease the chronic dose or enhance CYP2A6 inhibition. Furthermore, preexposure of an individual to one inhibitory substance can subsequently decrease the needed dose of a second inhibitor.

The appropriate dosage of a substance which inhibits CYP2A6 is dependent upon the amount of CYP2A6 that is present in an individual's body. This amount is in turn dependent upon whether the individual contains two variant alleles, one variant allele or no variant alleles at the CYP2A6 gene locus. A method for determining the CYP2A6 activity in an individual containing two variant alleles, one variant allele or no variant alleles at a gene locus for the CYP2A6 gene, is described in PCT Patent Application WO 99/27919.

The individual subject may be any type of mammal having a CYP2A6 enzyme. Suitably the subject is human. Generally, the subject is an individual having a CYP2A6 genotype associated with an active form of the enzyme. The CYP2A6 genotype of an individual and the existence of an active CYP2A6 enzyme in an individual may be determined using procedures described in PCT Patent Application WO 99/27919. For example, coumarin 7 hydroxylation has been used to measure CYP2A6 activity (Cholerton, et al. (1992); and Rautio, et al., (1992)). As discussed above, in an embodiment of the invention, the methods and compositions of the invention may be used in individuals or populations having high levels of CYP2A6, or in individuals where the behavioural components of smoking are significant.

For use in the treatment of conditions requiring regulation of nicotine metabolism to cotinine, by way of general guidance, a daily oral dosage of grape seed extract or its constituent can be about 0.01 to 80 mg/kg of body weight, specifically 0.1 to 20, more specifically 0.5 to 15 mg/kg of body weight. Ordinarily a dose of 0.7 to 11 mg/kg of a compound of the invention per day in divided doses one to multiple times a day, specifically up to four times per day, or in sustained release form is effective to obtain the desired results. In accordance with a particular regimen, grape seed extract or its constituents is administered once to four times daily for as long as necessary. While standard interval dose administration may be used intermittent dosing prior to high risk smoking times, e.g., early in the day and before the end of a working day may be beneficial.

More than one constituent of grape seed extract may be used to regulate metabolism of nicotine to cotinine. In such cases the substances can be administered by any conventional means available for the use in conjunction with pharmaceuticals, either as individual separate dosage units administered simultaneously or concurrently, or in a physical combination of each component therapeutic agent in a single or combined dosage unit. The active agents can be administered alone, but are generally administered with a pharmaceutical carrier selected on the basis of the chosen route of administration and standard pharmaceutical practice as described herein above.

The grape seed extract or its constituents may be administered alone or contemporaneously with nicotine and/or other inhibitors of CYP2A6 or other compounds which affect the metabolism of nicotine. In an embodiment of the invention, the compounds are administered contemporaneously with nicotine and/or inhibitors of CYP2B6. Inhibitors of CYP2B6 are described, for example in PCT Patent Application WO 99/27919. The pharmaceutical compositions and treatment methods may also be used together with other centrally active pharmaceutical compositions that modify smoking behaviour (e.g. bupropion, a.k.a. Wellbutrin™, in its various formulations) to decrease the dose of the centrally active composition or to increase its effectiveness in the treatment of tobacco dependence.

The following non-limiting examples are illustrative of the present invention:

### EXAMPLES

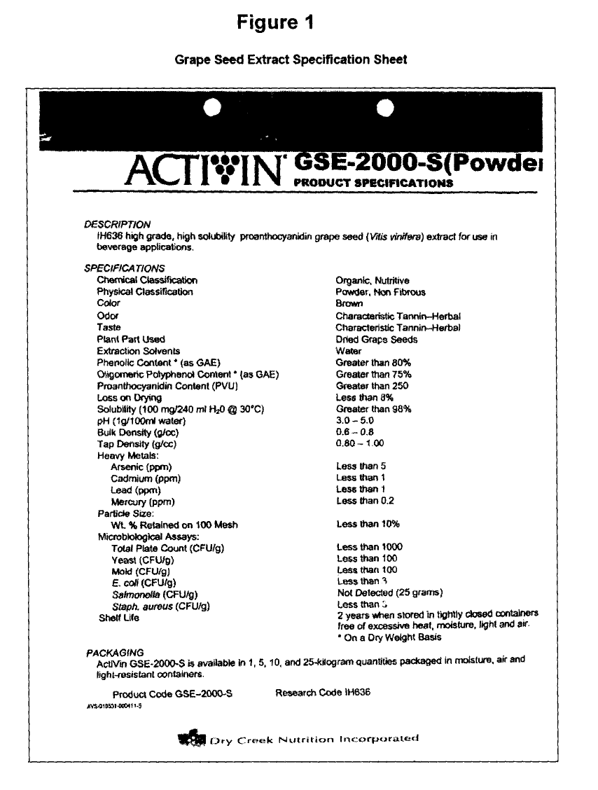

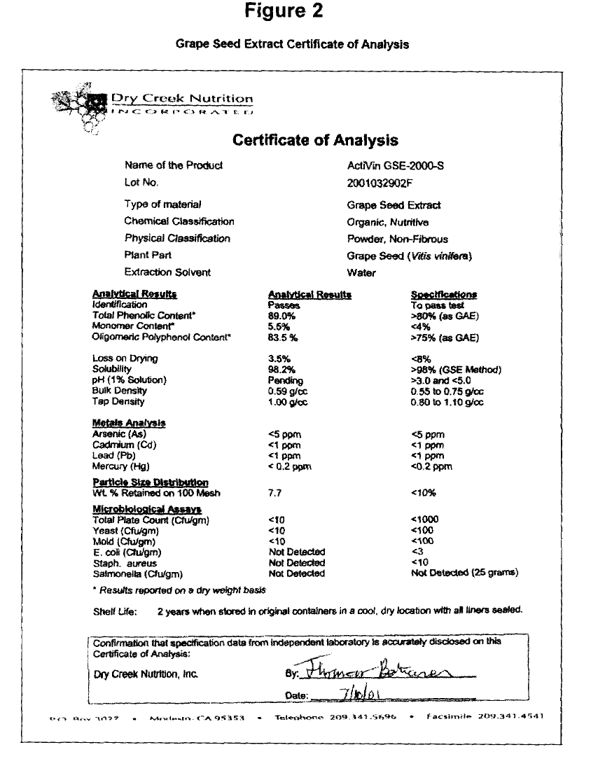

Grape seed extract (Activin® GSE-2000-S) lot number 2001032902F, was supplied by Dry Creek Nutrition of Modesto, Calif. (which is now San Joaquin Valley Concentrates of Fresno, Calif.) through NIEHS contract with Midwest Research Institute. The specification sheet and certificate of analysis are provided as FIGS. 1 and 2 respectively.

“Capsules” refer to a gelatin capsule each containing 650 mg of crushed grape seed. Capsules are available commercially as “Nature's Pearl Muscadine Grape Seed Capsules” from Nature's Pearl Inc., North Carolina.

### Example 1

### Preparation of Grape Seed Extract

Dry, cleaned Vitis vinifera grape seed (or any other species of the Genus Vitis, or any crossed hybrids, 400 pounds) are conveyed into a pressure vessel containing 300 gallons of deionized water. The mixer in the vessel is turned on and 25 pounds of bentonite is added into the vessel. The vessel is closed and the contents are heated at 240° F. for 2 hours. The mixture is then cooled and pumped over a parabolic screen to separate the seeds and some of the bentonite from the liquid extract. The extract is then filtered by a PCI ultrafiltration (UF) unit equipped with a FP-200 membrane at 80 gpm and 125° F. To facilitate filtration, additional water at approximately 37% of the extract volume is added (diafiltration) during the procedure. The permeate (about 1600 gallons) is then pumped into a separation column containing the adsorbent resin XUS-43520.00, a divinylbenzene copolymer manufactured by Dow Chemical, at 3 BV flow rate. The resin is then washed with 20% ethanol at 120° F. One thousand gallons of proanthocyanidins in water-alcohol (solution) is thus generated. The eluted proanthocyanidins are then concentrated by reverse osmosis using an ES404 membrane. Twenty gallons of concentrated proanthocyanidins extract is transferred into a vacuum dryer and dried at 175° F. and 29-30 mm Hg for six hours. Thirty-two pounds of dry proanthocyanidins extract is obtained and packaged for further grinding.

### Example 2

### Determination of the Catechin Content of Grape Seed Extract

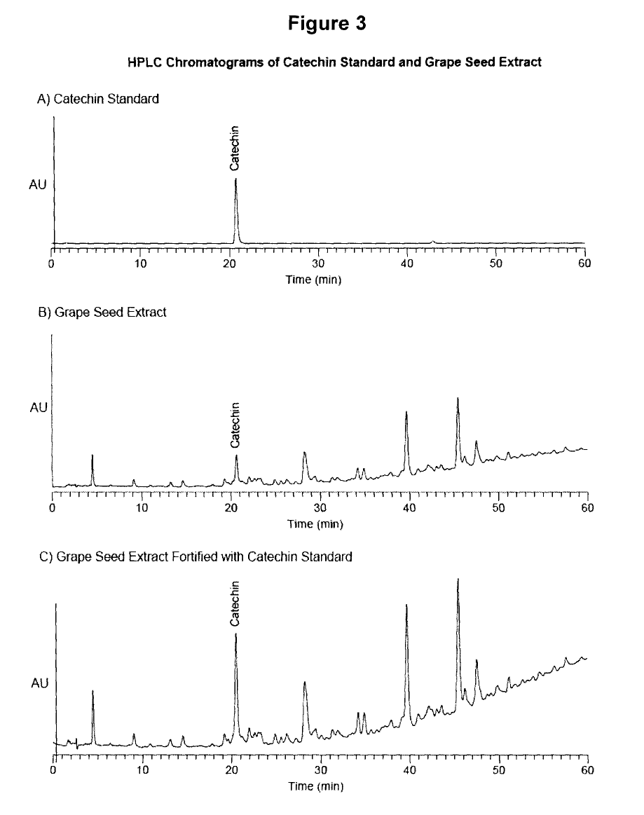

The grape seed extract was analyzed by HPLC to determine the relative concentration of catechin. The chromatographic system consisted of Phenomenex Luna™ Phenyl-Hexyl column and a mobile phase that increased linearly from 5% acetonitrile in 0.1% aqueous phosphoric acid to 75% acetonitrile in 0.1% aqueous phosphoric acid over 60 min. The flow rate was 1 ml/min. Catechin was detected and quantified with an Applied Biosystems 759A absorbance detector, using the integrated area of the absorbance at 278 nm. Standards were prepared using known concentrations of catechin, and a standard curve was calculated by linear regression analysis. Representative chromatograms of catechin standard and grape seed extract are presented in FIG. 3. Catechin accounted for 3.09% of grape seed extract.

### Example 3

### Determination of the Inhibitory Affect of Grape Seed Extract on Human Hepatic P450 Enzymes

The P450 enzyme assays were conducted on pooled samples of human liver microsomes (Lot Numbers HHM-0258) obtained from Tissue Transformation Technologies (TTT) of Edison, N.J. The pools were representative of the average enzyme activities found for the inventories of TTT human liver microsomes.

Treated incubation tubes were prepared by adding grape seed extract, dissolved in methanol, to each tube for a final assay concentration of 1, 3, 10, or 30 μM catechin. The methanol was allowed to evaporate. Control incubation tubes contained no grape seed extract. Microsomes and assay buffer were added to control and treated tubes as described below, and the tubes were preincubated at 37° C. for 10 min. NADPH was then added, and the incubations were maintained at 37° C. for an additional 15 min. Following initiation of the reactions by addition of substrate, assays were performed as described below.

### a) Determination of Microsomal Protein Concentration

Protein concentration was determined by a modified Bradford (Bradford, M. M. Anal. Biochem. 1976, 72:248-254) method using a DC Protein Assay kit (Bio-Rad, Hercules, Calif.) with bovine serum albumin (BSA) as a standard.

### b) Acetanilide 4-Hydroxylase Activity

Acetanilide hydroxylation, a marker for the activity of CYP1A2, was measured using a modified HPLC assay method of Liu et al. (Liu G. et al., Arch. Biochem. Biophys. 1991, 284, 400-406). The reaction mixture contained 50 mM Tris buffer (pH 7.5), 0.30 mM MgCl₂, 1 mM NADPH, 0.02 mg microsomal protein, and 0.1 mg of BSA in a volume of 100 μL. The reaction was initiated by the addition of 10 μL of 4 mM acetanilide. After 20 min of incubation at 37° C., the incubation was terminated by extraction with 0.5 mL of ethyl acetate. Extracts were removed, dried, reconstituted in mobile phase, and analyzed by reversed phase HPLC using a 4.6 mm×25 cm Zorbax™ SB-C18 column and an isocratic mobile phase of 90% aqueous KH₂PO₄ buffer (50 mM):10% acetonitrile. The flow rate was 1 mL/min, and the eluting hydroxyacetanilide was detected and quantitated by measuring the area under the UV absorbance curve at 254 nm. Standards were prepared using known concentrations of 4-hydroxyacetanilide, and a standard curve was calculated by linear regression analysis. Production of 4-hydroxyacetanilide in microsomal incubations was quantitated using regression variables and the area under the curve (AUC) for each incubation sample.

### c) Coumarin 7-Hydroxylase Activity

Coumarin 7-hydroxylation, a marker of CYP2A6 activity, was determined as described by Hickman et al. (Hickman D. et al., Drug Metab. Dispos. 1998, 26, 207-215) using a modified HPLC method. Reaction mixtures contained 100 mM potassium phosphate buffer (pH 7.4), 1 mM EDTA, 5 μM coumarin, and 0.01 mg microsomal protein in a final volume of 100 μL. Reactions were initiated by addition of 10 μL of 10 mM NADPH and incubated at 37° C. for 5 min prior to termination by the addition of 10 μL of 2 M HCl. Following centrifugation, an aliquot of each supernatant was analyzed by HPLC for 7-hydroxycoumarin production. Metabolites were separated on a Zorbax SB-C18 column using a flow rate of 0.8 mL/min and an isocratic mobile phase of 55% 20 mM sodium phosphate buffer (pH 4.4) and 45% methanol (v:v). The eluting 7-hydroxycoumarin was detected and quantitated by measuring the area under the UV absorbance curve at 324 nm. Standards were prepared containing known amounts of 7-hydroxycoumarin, and a standard curve was calculated by linear regression analysis. Production of 7-hydroxycoumarin was quantitated using regression variables and the AUC for each incubation sample.

Following incubation with grape seed extract normalized to 3 μM catechin, CYP2A6 activity decreased 63%. At catechin levels of 10 μM and greater, the CYP2A6 activity was decreased to a level below the lower limit of quantitation.

### d) Paclitaxel 6α-Hydroxylase Activity

Paclitaxel 6α-hydroxylation, a marker of CYP2C8 activity, was determined using a modified method of Harris et al. (Harris J W et al., Cancer Res. 1994, 54, 4026-4035). Reaction mixtures contained 100 mM potassium phosphate buffer (pH 7.4), 3.3 mM magnesium chloride, 1 mM NADPH, and 0.2 mg protein in a final volume of 0.25 mL. Reactions were initiated by the addition of paclitaxel (20 μM final concentration), prepared as a 5 mM stock in ethanol. The final concentration of ethanol in each incubation was 0.4%. Reactions were allowed to proceed for 10 min at 37° C. and were terminated by addition of 75 μL acetonitrile. Following centrifugation, an aliquot of each supernatant was analyzed by HPLC for 6α-hydroxypaclitaxel production. Metabolites were separated using a Zorbax™ SB-C18 column and a linear gradient from 60 to 70% aqueous methanol over 20 min at a flow rate of 1 mL/min. 6α-Hydroxypaclitaxel was detected and quantitated by measuring the area under the UV absorbance curve at 230 nm. Standards were prepared using known concentrations of 6α-hydroxypaclitaxel, and a standard curve was calculated by linear regression analysis. Production of 6α-hydroxypaclitaxel in microsomal incubations was quantitated using regression variables and the AUC for each incubation sample.

### e) Tolbutamide Hydroxylase Activity

Tolbutamide hydroxylation, a marker for the activity of CYP2C9, was measured using the method of Miners et al. (Miners J O., et al., Biochem. Pharmacol. 1988, 37, 1137-1144). The reaction mixture contained 0.1 M phosphate buffer, pH 7.4, 0.2 mg microsomal protein, and 1 mM NADPH in a final volume of 1 mL. Reactions were initiated by the addition of tolbutamide (2 mM final concentration) and incubated for 30 min at 37° C. The reactions were stopped by the addition of 1 mL of 0.15 M phosphoric acid. Next, 1.8 mL of the reaction mixture was extracted with 8 mL of 1000:250:5 hexane:chloroform:isoamyl alcohol to remove unreacted tolbutamide. The organic layer was removed and discarded. An internal standard (chloropropamide; 100 μL of a 10 mg/L solution) was added, and the samples were extracted with 8 mL of diethyl ether. The extracts were removed, dried, reconstituted in mobile phase, and analyzed by HPLC for 4-hydroxytolbutamide. A standard curve was generated similarly using known amounts of 4-hydroxytolbutamide. Samples were analyzed using a Zorbax™ Rx-C18 column with a mobile phase of 78:22 0.01 M sodium acetate buffer, pH 4.3: CH3CN and a flow rate of 2 mL/min. The metabolite was detected and quantitated by measuring the area under the UV absorbance curve at 230 nm. Standards were prepared using known concentrations of 4-hydroxytolbutamide, and a standard curve was calculated by linear regression analysis. Production of 4-hydroxytolbutamide in microsomal incubations was quantitated using regression variables and the area under the curve for each incubation sample.

Following incubation with grape seed extract normalized to 10 μM catechin the activity of CUP2C9 decreased 58%.

### f) (S)-Mephenyloin 4-Hydroxylase Activity

Mephenyloin 4-hydroxylation, a marker of CYP2C19 activity, was determined using the method of Hickman et al. (Drug Metab. Dispos. 1998, 26; 207-215) Reaction mixtures contained 100 mM potassium phosphate buffer (pH 7.4), 1 mM EDTA, 0.08 mg of microsomal protein, 1 mM NADPH, and 1 mM (S)-mephenyloin in a final volume of 0.1 mL. Reactions were initiated by addition of (S)-mephenyloin, prepared in acetonitrile (final concentration 5%), and incubated at 37° C. for 60 min prior to termination by addition of 10 μL of 2 M HCl.

Following centrifugation, an aliquot of each supernatant was analyzed by HPLC for the production of 4-hydroxymephenyloin. The chromatographic system consisted of a Supelcosil™ LC-18-DB column and a mobile phase of 3:2 water:methanol (v:v) at a flow rate of 1 mL/min. The eluting 4-hydroxymephenyloin was detected and quantitated by measuring the area under the UV absorbance curve at 230 nm. Standards were prepared containing known amounts of 4-hydroxymephenyloin, and a standard curve was calculated by linear regression analysis. Production of 4-hydroxymephenyloin was quantitated using regression variables and the AUC for each incubation sample.

g) p-Nitrophenol 4-Hydroxylase Activity

p-Nitrophenol (PNP) hydroxylation, a marker for the activity of CYP2E1, was measured using the method of Koop (Koop Dr. Mol. Pharmacol. 1986, 29, 399-404). The reaction mixture, maintained at 37° C., contained 1 mg microsomal protein, 1 mM ascorbate and 1 mM NADPH in a volume of 1 mL. Reactions were initiated by addition of PNP (0.1 M final concentration) and stopped after 6 min by addition of 200 μL ice cold 1.5 N HClO₄. Aliquots (1 mL) were removed following centrifugation and color developed by addition of 100 μL 10 N NaOH. The absorbance at 510 nm of each resulting solution was determined. Standards were prepared using known concentrations of 4-nitrocatechol, and a standard curve was calculated by linear regression analysis. Production of 4-nitrocatechol was quantitated using regression variables and the UV absorbance of each incubation sample.

### h) Dextromethorphan N-Demethylase Activities

Dextromethorphan O-demethylation, a marker for the activity of CYP2D6, was measured using the method of Hickman et al. (Drug Metab. Dispos. 1998, 26; 207-215) with a modification of the HPLC analysis method of Laurenzana et al. (Laurenzana E M et al., Drug Metab. Dispos. 1995, 23, 271-278). Dextromethorphan O-demethylase activity was measured at a substrate concentration of 20 μM dextromethorphan hydrobromide. The incubation mixtures also contained 0.02 mg microsomal protein, 0.1 M phosphate buffer, pH 7.4, and 1 mM NADPH in a final volume of 0.3 mL. The reactions were initiated by the addition of NADPH and were incubated at 37° C. for 30 min before they were stopped by the addition of 20 μL of 70% perchloric acid. After centrifugation, the supernatants were analyzed for dextromethorphan metabolites by HPLC using a Microsorb MV Phenyl™ column. The mobile phase was: A: 0.05 M phosphate buffer, pH adjusted to 4 with phosphoric acid, B: CH₃CN. The flow rate was 1.5 mL/min; the gradient started at 20% B and held at that composition for 8 min before changing linearly to 45% B over 3 min. Dextrorphan was detected and quantitated by fluorescence with the excitation and emission filters set at 270. A standard curve was generated using known amounts of metabolite standards.

Following incubation with grape seed extract normalized to 10 μM catechin the activity of CYP2D6 was decreased 46%.

### i) Laurate ω-Hydroxylase Activity

Lauric acid ω-hydroxylation, a marker for CYP4A11 activity, was measured by the method of Clarke et al. (Clarke S E et al., Chem Res. Toxicol. 1994, 7, 836-842). The incubation mixtures contained [¹⁴C]lauric acid (0.1 μCi, 20 mM), 0.6 mg microsomal protein in 50 mM Tris buffer (pH 7.4), and 0.44 mM NADPH in a final volume of 0.4 mL. The samples were preincubated at 37° C. for 3 min prior to initiation by the addition of lauric acid. The reactions were allowed to proceed for 5 min before termination by the addition of 200 μL 3 M HCl. The samples were extracted with 7 mL diethyl ether by end-over-end rotation for 20 min. The ether supernatants were removed, dried, and reconstituted in 150 μL of 45:55:5 water:methanol:acetic acid. Samples were analyzed by reversed phase HPLC using a Zorbax™ Rx-C18 column (4.6×250 mm) with the following mobile phase solutions: A: 0.2% aqueous acetic acid, and B: 0.2% acetic acid in methanol. The binary gradient employed to elute lauric acid, and its metabolites was at 48% A for the first 20 min of the run and then was changed linearly to 100% B over 3 min; after 7 min at 100% B, the composition returned to the initial composition over 1 min. The fraction containing ω-hydroxylated lauric acid was collected and analyzed by liquid scintillation spectrometry.

### j) Midazolam 1-Hydroxylase Activity

Midazolam 1-hydroxylation, a marker for the activity of CYP3A4, was measured using the method of Patki et al. (Patki K C et al., Drug Metab. Dispos. 2003, 31, 938-944). The reaction mixture contained 6 μM midazolam, 50 mM phosphate buffer (pH 7.4), 0.06 mg microsomal protein and 1 mM NADPH in a final volume of 0.25 mL. Reactions were initiated by the addition of midazolam and were allowed to proceed for 5 min at 37° C. before termination by the addition of 100 μL ice-cold acetonitrile. Following centrifugation, samples were analyzed for 1-hydroxymidazolam by reversed phase HPLC. The chromatography system consisted of a Zorbax™ Rx-C18 column with an isocratic mobile phase of 20:35:45 acetonitrile:methanol:10 mM potassium phosphate buffer, pH 7.4 and a flow rate of 1.4 mL/min. The 1-hydroxymidazolam metabolite was detected and quantitated by measuring absorbance at 220 nm. Standards were prepared containing known amounts of 1-hydroxymidazolam and a standard curve was calculated by using linear regression analysis. Production of 1-hydroxymidazolam was quantitated by using regression variables and the area under the UV absorbance curve for each incubation sample.

Following incubation with grape seed extract normalized to 3 μM catechin, Midazolam 1-hydroxylase was decreased 79%. In the presence of grape seed extract normalized at 10 μM, the enzyme activity was decreased below the lower limit of detection.

### k) Testosterone 6β-Hydroxylase Activity

Testosterone 6β-hydroxylase activity, a marker for the activity of CYP3A4, was measured by the method of Wood et al. (Wood A W et al., J. Biol. Chem. 1983, 258, 8839-8847). The reaction mixtures contained 50 μmol potassium phosphate buffer (pH 7.4), 25 μmol sucrose, 1.5 μmol MgCl₂, 0.5 mg microsomal protein, 125 nmol of testosterone (added in 5 μL of ethanol), and 0.5 μmol of NADPH in a final volume of 0.5 mL. The reaction was initiated by the addition of NADPH and incubated at 37° C. for 5 min prior to termination by the addition of 3 mL of ethyl acetate. After centrifugation, the organic layer was removed, the solvent was evaporated, and the residue was reconstituted in 40:60 methanol:H₂O and analyzed by HPLC for 6β-hydroxytestosterone. Extracts were analyzed by reversed phase HPLC using a 4.6 mm×150 mm Supelco ACE C18™ column. Testosterone metabolites were separated using a concave gradient consisting of methanol and water. The gradient started at 40% methanol and then increased over 30 min to 65% methanol, where it was held unchanged for an additional 15 min. The flow rate was 1 mL/min. The eluting 6β-hydroxytestosterone metabolite was detected and quantitated by measuring absorbance at 254 nm. Standards were prepared containing known amounts of 6β-hydroxytestosterone and a standard curve was quantitated by using regression variables and the area under the UV absorbance curve for each incubation sample.

Following incubation with grape seed extract normalized to 10 μM catechin, the activity at testosterone 6β-hydroxylase was decreased by 31%. Following incubation with Grape seed extract normalized at 30 μM catechin activity was diminished by 86%.

### l) 4.3.3.3 P-Glycoprotein ATPase Assay

Human P-glycoprotein (Pgp), expressed in a baculovirus expression system obtained from Gentest Corp. (Woburn, Mass.), was used for determination of verapamil-stimulated, vanadate-sensitive ATPase activity, an activity indicative of substrate binding to the active site of Pgp. Reaction mixtures contained TRIS-MES buffer (50 mM TRIS-MES, 2 mM EGTA, 50 mM KCl, 2 mM dithiothreitol and 5 mM sodium azide), 40 μg Pgp membranes and grape seed extract normalized to 0, 1, or 10 μM catechin in a final volume of 60 μL. Control ATPase reaction mixtures were prepared as described above with the addition of 100 μM sodium orthovanadate, an inhibitor of Pgp, to allow discrimination of non-Pgpmediated ATPase activity. Reactions were initiated by the addition of 0.24 μmol MgATP and incubated for 20 min at 37° C. prior to termination by the addition of 30 μL 10% SDS and Antifoam A. Following addition of 200 μL detection reagent (1:4 35 mM ammonium molybdate in 15 mM zinc acetate:10% ascorbic acid), reactions were incubated at 37° C. for an additional 20 min. The liberation of inorganic phosphate was quantitated by measuring absorbance at 630 nm and comparing with a phosphate standard curve.

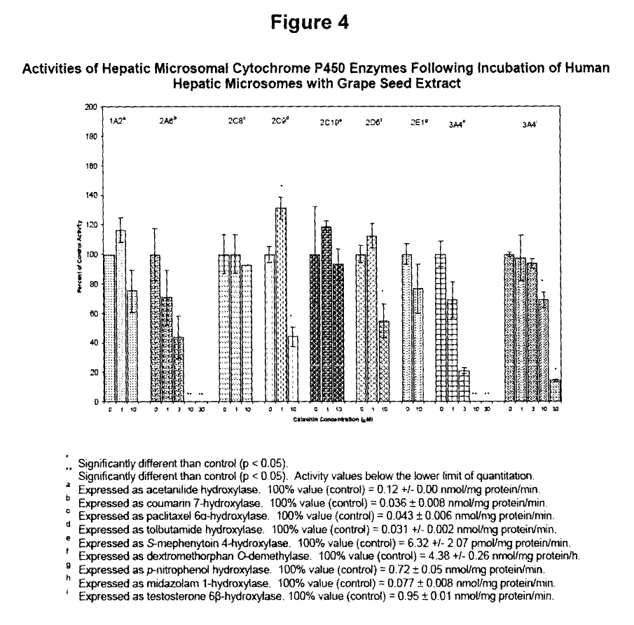

Results of the cytochrome P450 enzyme assay for various concentrations of catechin (μM) are shown in FIG. 4. The results show that grape seed extract normalized to 3 μM catechin resulted in significant inhibition of the activity of CYP2A6.

### Example 4

### Determination of the Percent Inhibition of Nicotine Metabolism in Mouse Liver Microsomes by GSE and Capsules Extracted in Buffer and Methanol

Samples of GSE and Capsules were prepared by extraction into buffer and into methanol. Buffer samples were extracted with ddH₂O, pH 4.1 (stomach acidity with food), at a concentration of 47 mg/ml for 2 hours at room temperature with continuous vortexing. Methanol samples were extracted at 47 mg/ml for 2 hours at room temperature followed by reconstitution with buffer at 47 mg/ml. The samples containing 0, 5, 50 or 500 μM of GSE or Capsule were incubated with adult male C57BI/6 hepatic microsomes pooled from two animals, with 0.5 mg/ml nicotine at 10×Km (100 μM) for 15 minutes at 37° C. The inhibitor concentration for Capsule samples was calculated based on the catechin content of the GSE, therefore the content of the active component in the Capsule samples will be far less than that in the GSE samples.

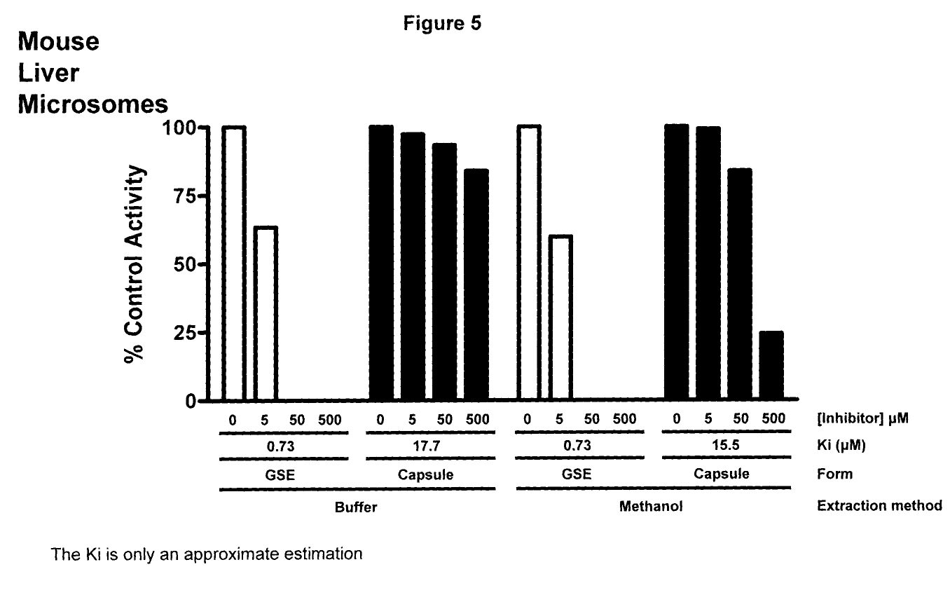

The results are shown in FIG. 5. In all cases a dose dependent inhibition of CYP2A6 in mouse liver microsomes was observed. As expected, due to the relative concentrations of the active substances, the GSE samples were more potent than the Capsule samples. Inhibition of CYP2A6 is mouse liver microsomes has been shown to be predictive of CYP2A6 metabolism in humans. Further, this data also shows inhibition of the metabolism of nicotine (vs. coumarin used as the substrate in Example 2).

### Example 5

### Determination of the Percent Inhibition in CYP2A6 Supersomes by GSE and Capsules Extracted in Buffer and Methanol

Samples containing 0, 5, 50, or 500 μM of GSE or Capsule were prepared as described in Example 4. Each sample was incubated with CYP2A6 Supersomes (human recombinant CYP2A6 in an artificial system comprising requisite factors for sample stability and function), with 20 pmol/ml nicotine at 100 μM, for 15 minutes at 37° C. As in Example 4 the inhibitor concentration for the Capsules was calculated based on the catechin content of the GSE and therefore had less active component compared to GSE.

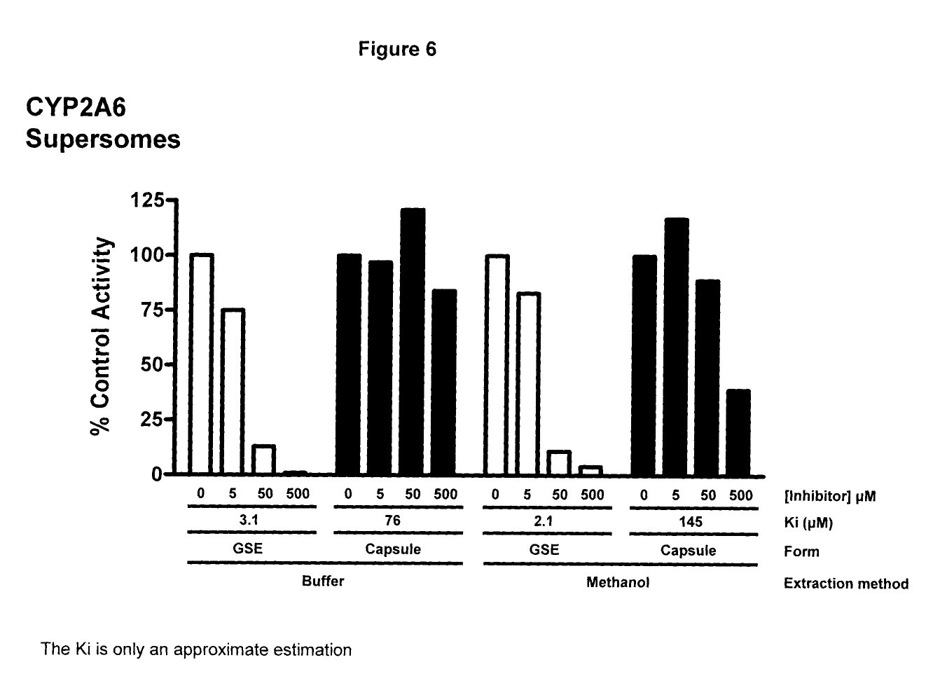

The results are shown in FIG. 6. Inhibition of CYP2A6 in Supersomes confirms that grape seed extract is the source for the inhibitors since, in this system, there is no possibility that another metabolite formed in human liver microsomes by reaction with another enzyme is acting as the inhibitor. Further, extraction of the grape seed extract and Capsule samples at pH 4.5, a pH that is similar to that in the stomach, indicates that the active compounds of grape seed extract will be extracted in the stomach.

### Example 6

### Control Experiment to Determine 100% Activity

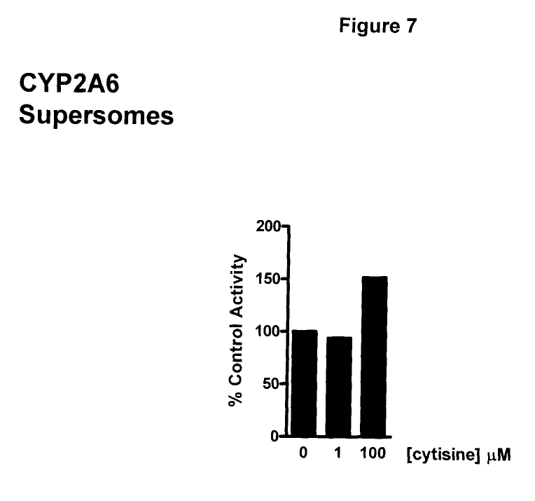

Sample containing 0, 1 and 100 μM of cytisine, a nicotine agonist, were prepared by extraction in ddH₂O pH 4.1, for 2 hours at room temperature with continuous vortexing. The extraction was done at a concentration of 47 mg/ml. The samples were incubated with CYP2A6 Supersomes with 20 pmmol/ml of nicotine at 100 μM for 15 minutes at 37° C. The results are shown in FIG. 7 and establish that a nicotine agonist does not have an inhibitory effect.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A method of preventing or treating conditions which benefit from an inhibition of CYP2A6 comprising administering an effective amount of grape seed extract or one or more of its constituent to a subject in need thereof.

2. The method according to claim 1, wherein the condition which benefits from an inhibition of CYP2A6 is one which benefits from an inhibition of nicotine metabolism to cotinine.

3. The method according to claim 2, wherein the condition that benefits from an inhibition of nicotine metabolism to cotinine is selected from nicotine-use disorders, nicotine-induced disorders and nicotine-related disorders.

4. The method according to claim 3, wherein the nicotine-use disorder is selected from dependent and non-dependent tobacco use, cardiovascular disease, pulmonary disease and drug dependency.

5. The method according to claim 4, wherein the nicotine-use disorder is dependent and non-dependent tobacco use or smoking.

6. A method according to claim 5, wherein the preventing or treating smoking is selected from:
a reduction in a likelihood of onset of smoking in a current non-smoking individual;
a reduction in a return to smoking of a previous smoker;
a reduction in an amount smoked by a current smoker;
a reduction in a use of tobacco products;
a reduction in a pattern of use of tobacco;
a reduction in an exposure to tobacco smoke;
a reduction in an amount of smoking which leads to cessation; and
a smoking cessation.

7. The method according to claim 3, wherein the nicotine-induced disorder is withdrawal from dependent and non-dependent tobacco use.

8. The method according to claim 3, wherein the nicotine related disorder is selected from opioid related disorders, proliferative disorders, cognitive disorders, neurological disorders and mental disorders.

9. The method according to claim 3, wherein the nicotine related disorder is selected from ulcerative colitis, Alzheimer's disease, Parkinson's disease, Tourette's Syndrome, sleep apnea, attention deficit disorder, Psychoses, schizophrenia, anxiety, depression, alcoholism, opiate dependence, memory deficits and cholinergic deficits.

10. The method according to claim 1, wherein the condition which benefits from an inhibition of CYP2A6 is one that is the result of activation of one or more procarcinogens by CYP2A6.

11. The method according to claim 10, wherein the condition that is the result of activation of procarcinogens by CYP2A6 is cancer.

12. The method according to claim 11, wherein the cancer is colorectal cancer.

13. The method according to claim 11, wherein the cancer is lung cancer.

14. The method according to claim 11, wherein the procarcinogen is a tobacco related nitrosamine.

15. A method of enhancing nicotine replacement therapy comprising administering an effective amount of grape seed extract or one or more of its constituents to a subject in need thereof.

16. The method according to claim 15, wherein the nicotine replacement therapy is administered by a method selected from oral, transdermal patch, nasal spray, and chewing gum inhalant.

17. The method according to claim 1, wherein the grape seed extract or one or more of its constituents is administered contemporaneously with nicotine.
